(19) United States
(12) Patent Application Publication
(10) Pub. No.: US 2009/0298938 A1
(43) Pub. Date: Dec. 3, 2009

(54) USE OF SEMI SYNTHETIC ANALOGUES OF BOSWELLIC ACIDS FOR ANTICANCER ACTIVITY

(75) Inventors: Ghulam Nabi Qazi, Jammu Tawi (IN); Subhash Chandra Taneja, Jammu Tawi (IN); Jaswant Singh, Jammu Tawi (IN); Ajit Kumar Saxena, Jammu Tawi (IN); Vijay Kumar Sethi, Jammu Tawi (IN); Bhahwal Ali Shah, Jammu Tawi (IN); Bal Krishan Kapahi, Jammu Tawi (IN); Samar Singh Andotra, Jammu Tawi (IN); Ajay Kumar, Jammu Tawi (IN); Shashi Bhushan, Jammu Tawi (IN); Fayaz Malik, Jammu Tawi (IN); Dilip Manikrao Mondhe, Jammu Tawi (IN); Shanmugavel Muthiah, Jammu Tawi (IN); Surjeet Singh, Jammu Tawi (IN); Monika Verma, Jammu Tawi (IN); Shashank Kumar Singh, Jammu Tawi (IN)

Correspondence Address: FULBRIGHT & JAWORSKI L.L.P., 600 CONGRESS AVE., SUITE 2400, AUSTIN, TX, 78701, US

(73) Assignee: COUNCIL OF SCIENTIFIC & INDUSTRIAL RESEARCH, New Delhi (IN)

(21) Appl. No.: 12/401,337

(22) Filed: Mar. 10, 2009

(30) Foreign Application Priority Data
Mar. 11, 2008 (IN) 606/DEL/2008

(51) Int. Cl. A61K 31/19; C07C 62/30

(52) U.S. Cl. 514/557; 562/498

(57) ABSTRACT

The present invention relates to use of compounds of general formula 1 for anticancerous activity, wherein the said compound being derived semi-synthetically from natural triterpenoic acids known as boswellic acids by the induction of apoptosis thereof cytotoxicity and anti-cancer activity displayed by semi-synthetic analogues of natural triterpenes, known as Boswellic acids. These compounds may be used for the treatment of cancer, alone or in combination with pharmaceutically acceptable or other carriers, displaying cytotoxicity and anti-cancer activity for colon, prostrate, liver, breast, central nervous system (CNS), leukemia and malignancy of other tissues, including ascites and solid tumors. The cancer cell death is mediated by induction of apoptosis and inhibition of cell proliferation at specific doses.

### BACKGROUND OF THE INVENTION

1. Field of the Invention

The present invention relates to use of compounds of general formula 1 for anticancer activity, being derived semi synthetically from natural triterpenoic acids known as boswellic acids. Cytotoxicity and anti-cancer activity displayed by semi-synthetic analogues of natural triterpenes, known as boswellic acids that may be used for the treatment of cancer, when used alone or in combination with pharmaceutically acceptable or other carriers. The compounds described herein display cytotoxicity and anti-cancer activity for colon, prostrate, liver, breast, central nervous system (CNS), leukemia and malignancy of other tissues, including ascites and solid tumors wherein the cancer cell death is mediated by induction of apoptosis and inhibition of cell proliferation at specific doses. These semi-synthetic boswellic acid analogues comprise alkyl acylates of a mixture of isomeric structures 3α-hydroxyurs-12-ene-24-oic acid, 3α-hydroxyolean-12-ene-24-oic acid, and 11-keto-3α-hydroxyurs-12-ene-24-oic acid, 11-keto-3α-hydroxyolean-12-ene-24-oic acid, and alkyl acylates of a mixture of epimeric structures 3β-hydroxyurs-12-ene-24-oic acid, 3β-hydroxyolean-12-ene-24-oic acid, 11-keto-3β-hydroxyurs-12-ene-24-oic acid, and 11-keto-3β-hydroxyolean-12-ene-24-oic acid. These semi-synthetic mixtures are prepared from the natural isolate of boswellic acid (α and β) or from semi-synthetic 11-keto-(α and β)-boswellic acid respectively.

2. Description of Related Art

The gum exudates from Boswellia serrata, the source of boswellic acids, have long been used in the traditional Ayurvedic system of medicine for the treatment of various inflammatory diseases on the Indian subcontinent, besides being used in preparations of commercial importance in the fragrance and cosmetic industries. The main triterpenoic acid constituents of the gum are boswellic acids represented by boswellic acid, acetyl boswellic acid, 11-keto-boswellic acids, acetyl-11-keto-boswellic acid (See FIG. 1A). Boswellic acid occurs in the form of an isomeric mixture i.e (α+β)-boswellic acids, similarly acetyl boswellic acid is also a mixture of isomeric acetyl (α+β)-boswellic acids.

Detailed pharmacology of the acid fraction comprising mainly boswellic acids has demonstrated dose related anti-inflammatory activity in various test models. Various publications have appeared in the past related to anti-inflammatory and related activities of boswellic acids as a mixture or individually [Roy et al., 2005; Ammon et al., 1993 and 2002; Gupta et al., 1997 and 2001; Kreiglstein et al., 2001; Schweizer et al., 2000; Safayhi et al., 1992]. A boswellic acid mixture was also found to be effective in adjuvant arthritis and to possess anti-complimentary activity [Sharma et al., 1989; Kapil et al., 1992].

The anti-cancer related activities of Boswellia serrata and its isolates has drawn the attention of scientists working in this area, and several reports on this subject have appeared in last one decade and a half. Second to coronary heart diseases, cancer has emerged as the most common cause of death in many countries. Unlike normal cells, cancer cell often appear after having undergone an accumulation of gene mutations which may initiate an independent and uncontrolled growth of cancer cells into a clonal expansion that results in an invasion of adjoining tissues, and sometimes, metastasis to other tissues. Dysregulation of programmed cell death (apoptosis) is a hallmark of all cancer cells and hence provides important therapeutic target to intervene in the development of anti-cancer agents. Accordingly, activation of apoptosis is a common cytotoxic mechanism employed by anti-cancer agents [Debatin, 2000]. Several apoptotic pathways are proposed to be involved in the death of cancer cells by anti-cancer drugs. An anti-cancer drug may kill cells by activation of death receptors mediated pathways or via mitochondrial dependent pathways involving reactive oxygen and nitrogen species culminating in activation of certain genes in the apoptotic signal transduction pathways. Several agents are known to kill malignant cells by inducing apoptosis via free radicals generation and one such example is doxorubicin [Minotti et al., 2004]. Most of these agents activate various caspases, genes, translocate cytochrome c that upregulate downstream events leading to DNA fragmentation (DNA laddering) typical of apoptosis.

In the next two decades, the world is expected to peak in all kinds of cancer, and brain cancer in particular appears difficult to manage. It is believed that there is a higher chance of discovering a new anti-cancer drug form natural sources than from synthetic programs. Presently 70% of the anti cancer drugs are either natural products or based on a natural molecule. Boswellic acids, the natural triterpenic acids from Boswellia sp., are reported to promote apoptosis in various cancer cells and work in this area is well documented [Syrovets et al., 2005; Liu et al., 2002; Zhao et al., 2003; Jing et al., 1999; Glaser et al., 1999; Hoernlein et al., 1999; Hostanska et al., 2002].

There has been a report of inhibitory activity of boswellic acids against human leukemia HL-60 cells in culture. The effect of acetyl-11-keto-β-boswellic acid on DNA synthesis was found to be irreversible. This molecule also significantly inhibits the cell growth of HL-60 [Shao et al., 1999]. Reports have claimed the DNA topoisomerase I and II inhibitory effect by pentacyclic triterpenic acids of Boswellia serrata [Lee et al., 1991; Syrovets et al, 2000]. There have also been reports on the use of boswellic acid in the treatment of brain tumors [Simmet Thomas (De) (1994) U.S. Pat. No. 5,919,821 and U.S. Pat. No. 6,174,876; Janssen G, et al., 2000; Streffer, J. R. et al., 2001]. Other anti-cancer related activities of boswellic acids also include increased leucocytic elastase activity [Ammon, et al. use of boswellic acid and its derivatives for inhibiting normal and increased leucocytic elastase or plasmin activity, U.S. Patent Application 2002/0010168].

The process of preparation of some of these semi-synthetic analogues has been the subject of various patents [See Indian Patents 178627, 180492, and 178627]. However, these analogues have only been reportedly screened for anti-inflammatory and related bio-activities however, use of the semi-synthetic analogues of boswellic acids of formula 1 for cancer cell related cytotoxity and apoptosis thereof is novel.

It is apparent from the review of literature on boswellic acids that most references report the use of either the extracts comprising boswellic acids or individual natural boswellic acids for their cytotoxic and anticancer related activities. There has not been any report on the bioactivity of semi-synthetic analogues of boswellic acids for the modulation cancer related disorders. Therefore, the use of the semi-synthetic analogues for the induction of apoptosis thereof cytotoxicity is novel.

Accordingly, the present invention addresses a screening of the bioactivity of semi-synthetic analogues boswellic acids of formula 1 as cytotoxic vis-à-vis pro-apoptotic agents, and demonstrates an improvement in efficacy represented by these analogs which may be useful for the treatment of cancer derived from various tissues. In addition, the present invention addresses developing a preparation or a formulation comprising semi-synthetic analogues of boswellic acids for their use as cytotoxicity related anti-cancer agents.

### SUMMARY OF THE INVENTION

The present invention relates to the induction of apoptosis thereof cytotoxicity and anticancer activity by semi-synthetic analogues of boswellic acids of the formula 1 alone or in combination with pharmaceutically acceptable or other carriers for cancers of colon, prostrate, liver, breast, central nervous system (CNS), leukemia and malignancy of other tissues, including ascites and solid tumors wherein the cancer cell death is mediated by induction of apoptosis and inhibition of cell proliferation at specific doses of the compound. The present invention also demonstrates the efficacy of selected semi-synthetic analogues boswellic acids of formula 1 as cytotoxic and pro-apoptotic agents, and provides a preparation comprising semi-synthetic analogues of boswellic acids for their use as cytotoxicity related anti-cancer agents.

Accordingly, the present invention provides the use of compounds of general formula 1 for anticancer activity, wherein the said compound being derived semi-synthetically from natural triterpenoic acids known as boswellic acids, wherein R₁ is H or Me; R₂=R₃=H or acyl group with C₂-C₅ alkyl chain; R₄=R₅=H or R₄+R₅=O; R₆=R₇=H or Me.

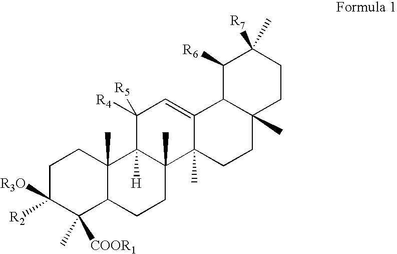

In an embodiment of the present invention, the representative compounds of the general formula 1 comprise:

3α-hydroxyurs-12-ene-24-oic acid;

3α-hydroxyolean-12-ene-24-oic acid

11-keto-3α-hydroxyurs-12-ene-24-oic acid;

11-keto-3α-hydroxyolean-12-ene-24-oic acid

3β-hydroxyurs-12-ene-24-oic acid;

3β-hydroxyolean-12-ene-24-oic acid

11-keto-3β-hydroxyurs-12-ene-24-oic acid

11-keto-3β-hydroxyolean-12-ene-24-oic acid.

In another embodiment of the present invention, the said compounds show cytotoxicity and anti-cancer activity up to 100% for colon, prostate, liver breast, central nervous system (CNS), leukemia and malignancy of other tissues, kidney, lung, muscle, ovarian and prostate cancer.

In yet another embodiment of the present invention, the said compounds further show cytotoxicity and growth inhibition up to 52% of Ehrlich Ascites tumors and solid tumors.

In yet another embodiment of the present invention, the said compounds kills up to 99% of prostrate cancer cells selected from DU-145 and PC-3 cell lines at 5×10⁻⁵M concentration.

In still another embodiment of the present invention, the said compounds kills colon cancer cells selected from HT-29, SW-620 and Colo205 cell lines up to 101% at 5×10⁻⁵M concentration.

In still another embodiment of the present invention, the said compounds inhibits growth of cancer cells of liver. Hep2 up to 100% at 5×10⁻⁵M concentration.

In further another embodiment of the present invention, the cancer cell death is mediated by induction of apoptosis and inhibition of cell proliferation.

In further another embodiment of the present invention, the said compounds being capable of producing early reactive nitrogen species nitric oxide up to 80% and acting as causative agents ensuing DNA laddering and apoptotic death of cancer cells.

In still further another embodiment of the present invention, the said compounds being capable of inducing at least up to 50% mitrocondrial depolarization consequent to reactive oxygen/nitrogen species as the mechanisms in to the apoptotic death of cancer cells.

In further another embodiment of the present invention, the concentration of the compound of general formula 1 used in the composition being in the range of 5-100 μg/ml.

In another embodiment of the present invention, the therapeutically effective dose of the said composition is in the range of 5×10⁻⁵-100×10⁻⁵ M.

In still another embodiment of the present invention, the therapeutically effective dosage of the said composition ranging between 5×10⁻⁵-100×10⁻⁵ M for 5-15 days to be administered to a subject.

In still further another embodiment of the present invention, the subject used is mammal including human.

In another embodiment of the present invention, the route of administering being selected from the group consisting of oral, intra-venous, intra-peritoneal, nasal.

In another embodiment of the present invention, the said composition being non-toxic in the range of 250-1000 μg/kg body weight of the subject.

### BRIEF DESCRIPTION OF THE DRAWINGS

FIGS. 1A-1B. FIG. 1A depicts the main triterpenoic acid constituents of the gum exudates from Boswellia serrata, the source of boswellic acids, represented by boswellic acid, acetyl boswellic acid, 11-keto-boswellic acids, acetyl-11-keto-boswellic acid. FIG. 1B shows the results of an examination of cell proliferation in HL-60 cells after 48 hr of treatment with indicated concentrations of structural analogs of boswellic acid. Cell proliferation analysis was performed in HL-60 cells using the MTT method described in the Examples section. Cells were incubated with each formula 1 analog for 48 hours at indicated concentration and thereafter, the mitochondiral competence of MTT reduction as an index of cell viability was determined. The optical density (OD) of the untreated control was taken as 100% viability. The analogs inhibited cell proliferation with an average IC50 of about 12 μM. Compound of formula 1, where R₃=COCH₂CH₂CH₃, R₁=H, R₄+R₅=O however, appeared to exert marginally stronger effect compared to other analogs. This analog, as a prototype, was selected for detailed investigations. Where a=compound of formula 1, R₃=COCH₂CH₂CH₃, R₁=H, R₄=R₅=H, b=compound of formula 1, R₃=COCH₂CH₃, R₁=H, X=H₂, c=compound of formula 1, R₃=COCH₂CH₂CH₃, R₁=H, R₄+R₅=O, d=compound of formula 1, R₁=H, R₃=COCH₂CH₃, R₄=R₅=H, e=compound of formula 1, R₃=COCH₂CH₂CH₃, R₁=H, R₄=R₅=H, f=β-boswellic acid

FIG. 2. Comparative efficacy of apoptosis induction: Measurement by flow cytometry. The effects of boswellic acid on the relative efficiency of induction of apoptosis and necrosis in HL-60 cells were analyzed by flow cytometry. Cells were incubated with each analog for 6 hrs and stained with annexin V-FITC labeled. The dot plots are representative of one of the two separate experiments. Values in the lower right quadrant represent the apoptotic population in the UR as post-apoptotic and in the UL as necrotic. Where a=compound of formula 1, R₃=COCH₂CH₂CH₃, R₁=H, R₄=R₅=H, b=compound of formula 1, R₃=COCH₂CH₃R₁=H, R₄=R₅=H, c=compound of formula 1, R₃=COCH₂CH₂CH₃, R₁=H, R₄+R₅=O, d=compound of formula 1, R₁=H, R₃=COCH₂CH₃, R₄=R₅=H, e=compound of formula 1, R₃=COCH₂CH₂CH₃, R₁=H, R₄=R₅=H, f=β-boswellic acid. Compounds of formula 1 where R₁=H, R₃=COCH₂CH₃R₃=R₄=H; R₃=COCH₂CH₂CH₃, R₁=H, R₄=R₅=H and natural β-boswellic acid produced low level of apoptotic cell population (approx. 10%) compared to compound of formula 1 where R₃=COCH₂CH₂CH₃, R₁=H, R₄=R₅=H (17%), R₁=H, R₃=COCH₂CH₃, R₄=R₅=H (30%) and where R₃=COCH₂CH₂CH₃, R₁=H, R₄+R₅=O (23%), although the combined apoptotic and post-apoptotic population was about 32% with the last three analogs. Confirmation that boswellic acid analogs are potent inducers of apoptosis may also be obtained from DNA fragmentation assay.

FIG. 3. Induction of DNA laddering in HL-60 cells by structural analogs of Boswellic acid

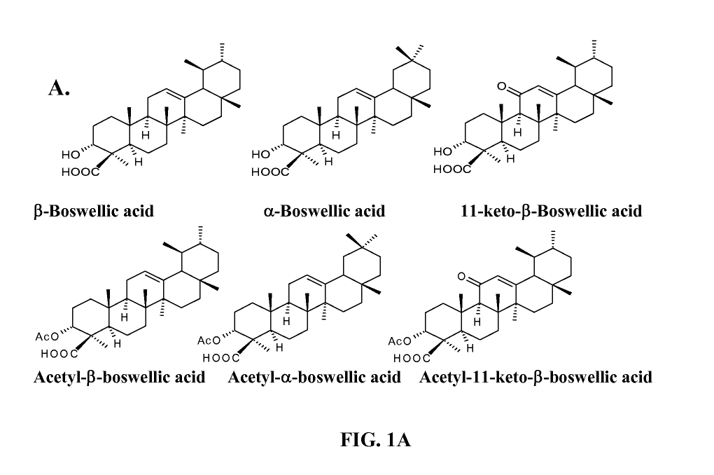

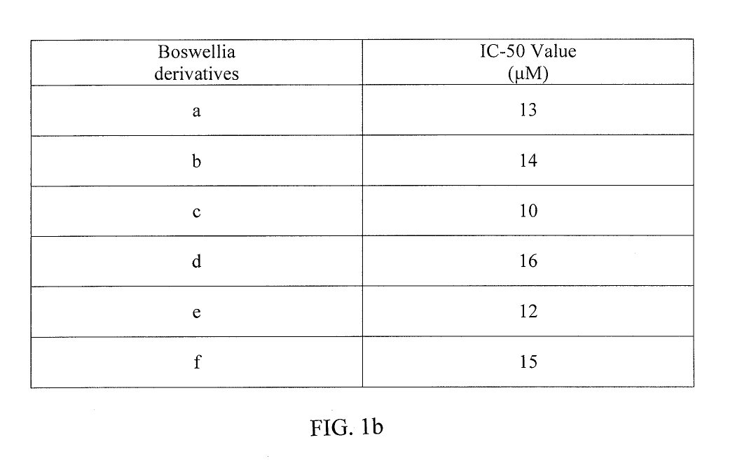

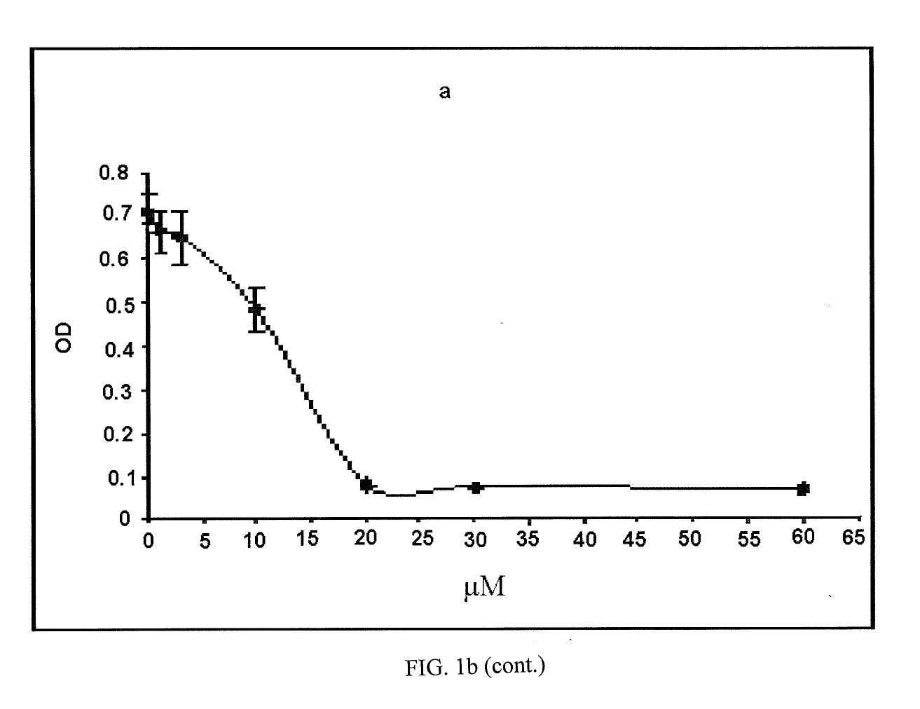

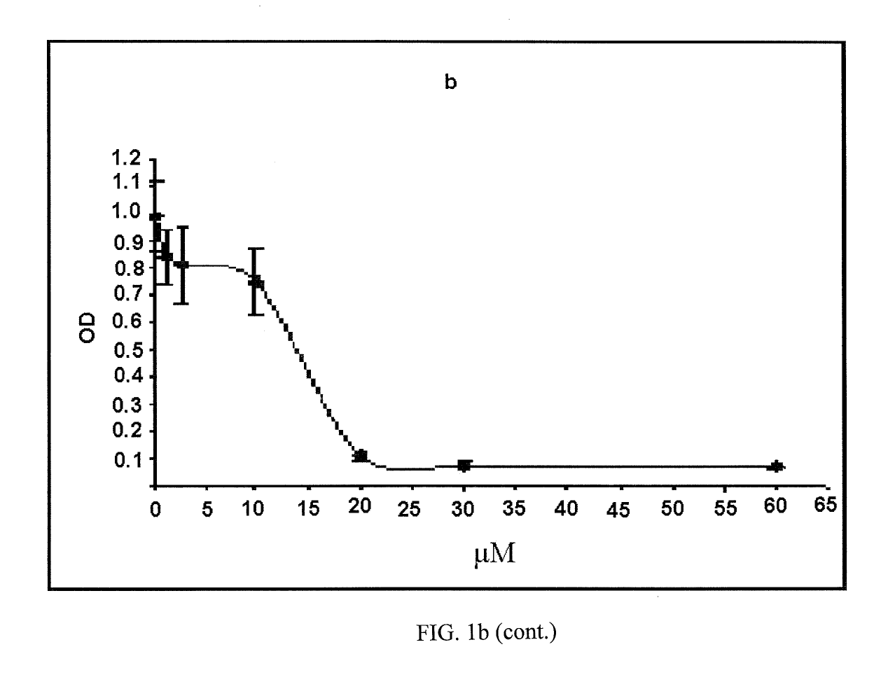

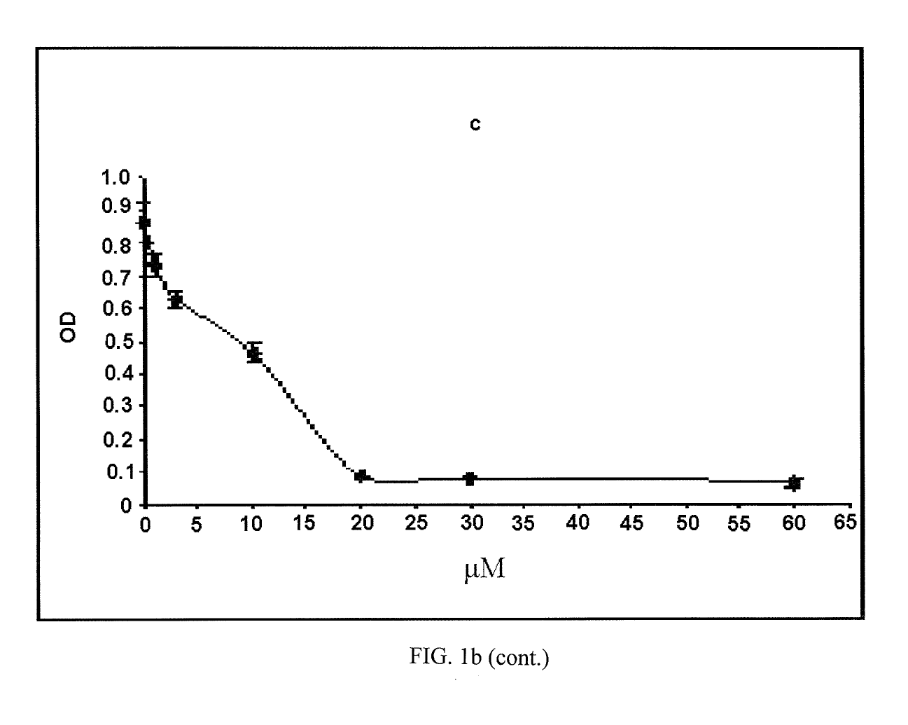

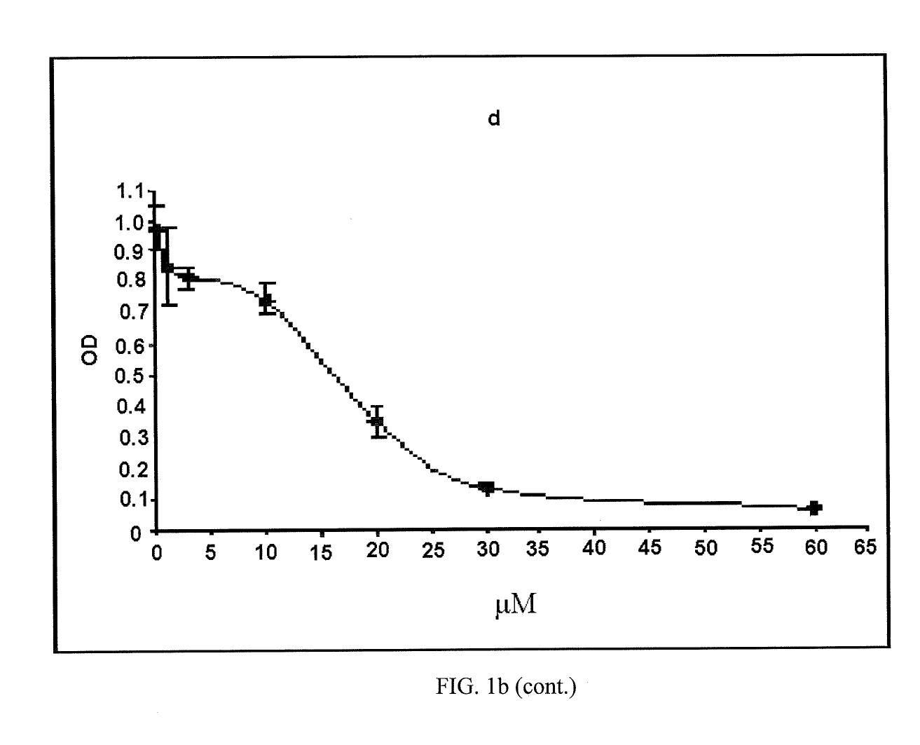

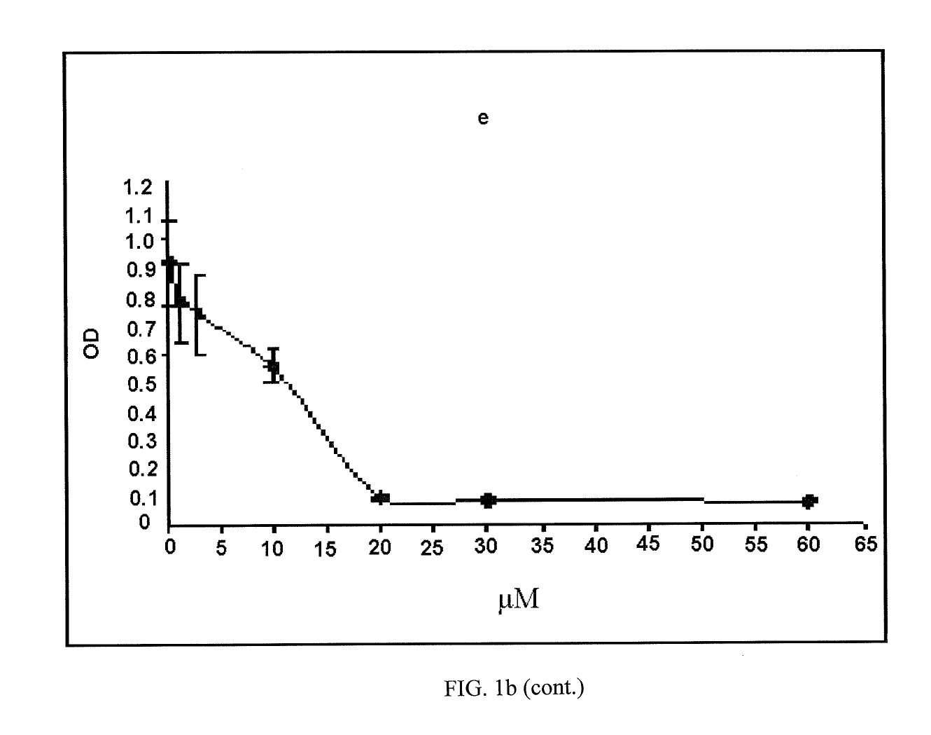

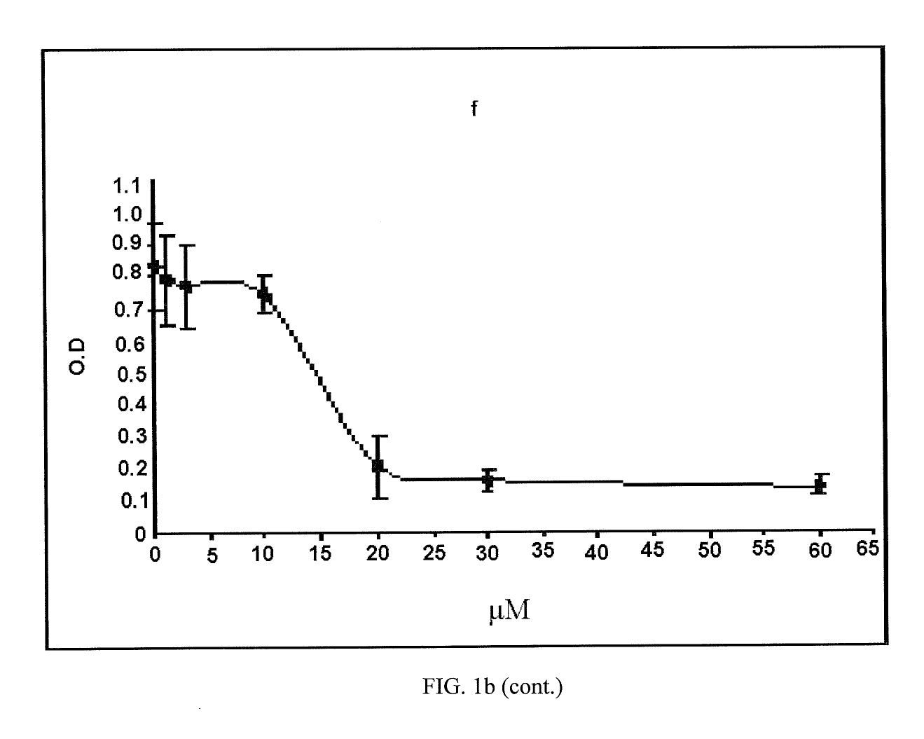

Apoptotic DNA ladder was observed when 2×106 HL-60 cells were treated with various analogs of boswellic acid at indicated concentrations for 6 hours, and DNA was extracted and electrophoresed on 1.8% agarose gel. Where, a=compound of formula 1, R₃=COCH₂CH₂CH₃, R₁=H, R₄=R₅=H, b=compound of formula 1, R₃=COCH₂CH₃, R₁=H, R₄=R₅=H, c=compound of formula 1, R₃=COCH₂CH₂CH₃, R₁=H, R₄+R₅=O, d=compound of formula 1, R₁=H, R₃=COCH₂CH₃, R₄=R₅=H, e=compound of formula 1, R₃=COCH₂CH₂CH₃, R₁=H, R₄=R₅=H, f=β-boswellic acid. All the compounds tested produced DNA fragmentation typical of apoptosis, suggesting that the boswellic acid analogs may induce cell death by apoptosis, a desired molecular target in the development of anti-cancer therapeutics.

FIG. 4. Boswellic acid analogs produce early generation of reactive oxygen species. In order to determine the intracellular content of intracellular peroxides, HL-60 cells in both treated and untreated control groups of 1×10⁶ cells were incubated with 5 mM of DCFH-DA for 30 min prior to termination of a 6 hr treatment with the indicated concentrations of compound designated “c” which is the compound of formula 1, wherein R₃=COCH₂CH₂CH₃, R₁=H, R₄+R₅=O. Treatment with the compound designated “c” was associated with an increase in DCFH-DA derived fluorescence. The compound of formula 1 where R₃=COCH₂CH₂CH₃, R₁=H, R₄+R₅=O was taken as a prototype to investigate the mechanism of cell death mediated by induction of apoptosis. This compound produced concentration related increase in ROS generation. At the highest concentration used the peroxide generation was relatively of low order because of ROS associated cell death. BSO was used as positive control.

FIG. 5. Copious early endogenous formation of nitric oxide by compound of formula 1 where R₃=COCH₂CH₂CH₃, R₁=H, R₄+R₅=O in HL-60 cells. Nitric Oxide (NO) production was measured by flow cytometry in the FL1 channel using DAF-2-DA (an NO probe) after exposing HL-60 cells for 4 hours to indicated concentrations of SS-145. To investigate the contribution of iNOS to NO production, cells were pre-incubated with iNOS inhibitor sMIT (0.5 mM) for 1 hour prior to treatment with a compound designated “c” (a compound of formula 1 wherein R₃=COCH₂CH₂CH₃, R₁=H, R₄+R₅=O). NO and peroxide generation are the early events leading to oxidative stress in the cells. Compound of formula 1 where R₃=COCH₂CH₂CH₃, R₁=H, R₄+R₅=O produced early generation of NO (FIG. 5) parallel to the production of ROS. It is possible that NO stimulated mitochondrial superoxide generation which together with NO may form peroxynitrite, a dangerous species in producing oxidative damage to the cells and responsible for production of apoptosis

FIG. 6. Dysregulation of mitochondrial membrane potential by compound of formula 1, where R₃=COCH₂CH₂CH₃, R₁=H, R₄+R₅=O in HL-60 cells. The effect of compound of formula 1, wherein R₃=COCH₂CH₂CH₃, R₁=H, R₄+R₅=O, on the membrane potential (Δψ mt) was analyzed in HL 60 cells by flow cytometry. Cells were incubated for 12 hours with compound “c”, and rhodamine 123 (5 μM) was added 30 min prior to the termination of treatment. Propidium iodide (5 μg/mL) was added prior (5 min) to flow cytometry analysis. A 20 uM concentration of compound “c” was able to reduce the transmembrane potential by about 42% suggesting the reactive oxygen and nitrogen species produced depolarization of mitochondrial membranes allowing the release of proapoptotic factors into the cytosol.

FIG. 7. Modulation of reduced glutathione contents in HL-60 cells by boswellic acid analogs. GSH content was measured in HL-60 cells as described in the Examples section, using treated and untreated groups of about 3×10⁶ cells per well and 3 mL of RPMI media. Cells were treated with buthionine sulfoxime (BSO), N-acetyl cysteine (NAC), a compound designated “c” (formula 1 compound wherein R₃=COCH₂CH₂CH₃, R₁=H and R₄+R₅=O), and NAC+the compound “c”. Treatment concentrations were as indicated. Compound of formula 1 where R₃=COCH₂CH₂CH₃, R₁=H, R₄+R₅=O strongly depleted GSH content, which was dose dependent. The depleted GSH stores were restored by an anti-oxidant n-acetyl cysteine. Depletion of GSH content is an important indicator of predisposition conditions leading to cellular injury. Loss of mitochondrial functions as a result of GSH depletion may affect the energy status of the cells and therefore, is critical event in the induction of cell injury. GSH in general offers protection by scavenging reactive oxygen species and electrophiles formed within the cells. The GSH content of cells recovered back to normal or NAC control when cells were treated with sulfhydral groups antioxidant, N-acetyl cysteine (NAC). This suggests clearly that boswellic acid analogs produced oxidative stress in cells, which may also activate caspases, cysteine-aspartyl proteases.

FIGS. 8A-8B. Caspase activation in HL-60 cells by boswellic acid analogs. FIG. 8A depicts the results of a colorimetric evaluation of caspase-3 activation in HL-60 cells and Molt-4 cells. Cells were treated with a compound designated “c” (formula 1 compound wherein R₃=COCH₂CH₂CH₃, R₁=H and R₄+R₅=O) at concentrations indicated for 6 hours. Caspase-3 inhibitor (DEVD-fmk) was added 30 min prior to the addition of caspase-3 substrate (DEVD-pNA) to the lysate. Data are mean of triplicate determinations with coefficients of variation less than 10%. FIG. 8B shows caspase-9 activation in HL-60 cells by a compound of formula 1 wherein R₃=COCH₂CH₂CH₃, R₁=H and R₄+R₅=O. HL-60 cells were treated for 6 hours with camptothecin (4 μM), etoposide (10 μM) as positive control, or with a compound designated “c” (formula 1 compound wherein R₃=COCH₂CH₂CH₃, R₁=H and R₄+R₅=O). Caspase-9 inhibitor LEHD-CHO was added 30 min prior to the addition of caspase-9 substrate to validate the specificity of caspase-9. Caspase-9 activity was measured fluorometrically. Data are a mean of triplicate determinations with a coefficient of variation of less than 10%.

FIG. 9. Tumor growth inhibition by boswellic acid analogs. Mice treated intraperitoneally with a compound (designated “c”) of formula 1, wherein R₃=COCH₂CH₂CH₃, R₁=H, R₄+R₅=O, have shown 22% tumor growth inhibition at a dose of about 50 mg/kg of body weight, and 41% tumor growth inhibition at a dose of 100 mg/kg of body weight. The positive control 5-FU (22 mg/kg body weight.) showed 56% tumor growth inhibition with respect to normal saline treated animals.

### DETAILED DESCRIPTION OF THE INVENTION

The compounds of formula 1 display cytotoxicity and anti-cancer activity against cancers of the colon, prostrate, liver, breast, central nervous system (CNS), leukemia, as well as malignancies of other tissues, including ascites and solid tumors, wherein the cancer cell death is mediated by induction of apoptosis and inhibition of cell proliferation at specific doses. These semi-synthetic boswellic acid analogues of formula 1 comprise alkyl acylates of a mixture of isomeric structures 3α-hydroxyurs-12-ene-24-oic acid, 3α-hydroxyolean-12-ene-24-oic acid, and 11-keto-3α-hydroxyurs-12-ene-24-oic acid, 11-keto-3α-hydroxyolean-12-ene-24-oic acid, and alkyl acylates of a mixture of epimeric structures 3β-hydroxyurs-12-ene-24-oic acid, 3β-hydroxyolean-12-ene-24-oic acid, 11-keto-3β-hydroxyurs-12-ene-24-oic acid, and 11-keto-3β-hydroxyolean-12-ene-24-oic acid. These semi-synthetic mixtures are prepared from the natural isolate of boswellic acid (α and β) or from semi-synthetic 11-keto-(α and β)-boswellic acid respectively.

In an aspect, the bioactivities of semi synthetic analogues boswellic acids of formula 1 are disclosed herein as cytotoxic and pro-apoptotic activities. As such, these compounds may be useful for the treatment of cancer derived from various tissues. A preparation or formulation is also provided comprising semi-synthetic analogues of boswellic acids for their use as cytotoxicity related anti-cancer agents.

The semi-synthetic analogues of formula 1 have been subjected to in vitro cytotoxicity screening in a panel of human cancer cell lines using following methodology. The human cancer cell lines were obtained either from National Center for Cell Science, Pune or National Cancer Institute, Fredrick, Md., USA. Cells were grown in tissue culture flasks in complete growth medium (RPMI-1640 medium with 2 mM glutamine, 100 g/ml streptomycin, pH 7.4, sterilized by filtration and supplemented with 10% sterilized fetal calf serum and 100 units/ml penicillin before use) at 37° C. in an atmosphere of 5% CO₂ and 90% relative humidity in a carbon dioxide incubator. The cells at sub-confluent stage were harvested from flask by treatment with trypsin (0.05% trypsin in PBS containing 0.02% EDTA) and suspended in complete growth medium. Cells with cell viability of more than 97% by trypan blue exclusion technique were used for determination of cytotoxicity.

Test material were dissolved in DMSO (dimethyl sulphoxide) to obtain a stock solution of 2×10⁻²M. The stock solution was serially diluted with complete growth medium containing 50 μg/ml of gentamycin to obtain working test solutions of required concentrations.

The suspension of human cancer cell lines of required cell density in complete growth medium was prepared and cell suspension (100 μl/well) of each cell line was added to wells of 96-well tissue culture plate. Suitable blanks and controls were also included. The plates were incubated for 24 hrs in a carbon dioxide incubator.

The working test solutions of different concentrations (100 μl/well) were added after 24 hr incubation. The plates were further incubated for 48-hrs after addition of the test materials. After incubation, the cell growth was stopped by gently layering of 50 μl of TCA (50% trichloroacetic acid) on top of the medium in all the wells. The plates were incubated at 4° C. for one hour to fix the cells attached to bottom of the wells. Liquids of all the wells were gently pipetted out and discarded. The plates were washed five times with distilled water to remove TCA, growth medium, low molecular weight metabolites, serum proteins etc. The plates were air dried.

Cell growth was measured by staining with sulforhodamine B dye (SRB). The SRB solution (100 μl, 0.4% in 1% acetic acid) was added to each well and the plates were incubated at room temperature for 30 minutes. The unbound SRB was quickly removed by washing the wells five times with 1% acetic acid solution and plates were air dried. Tris buffer (100 μl, 0.01 M, pH 10.4) was added to all the wells and plates were gently stirred for 5 minutes on a mechanical stirrer. The optical density was recorded on ELISA reader at 540 nm.

Cell growth in the presence of test material was calculated in terms of the percentage of growth inhibition. The in vitro cytotoxicity of four natural boswellic acid i.e. boswellic acid, acetyl boswellic acid, 11-keto-boswellic acids, acetyl-11-ketio-boswellic acid on various cancer cell lines has been examined and typical results are given in the succeeding table. Based on their cytotoxicity profile the analogues of formula 1 were studied for inhibition of cell proliferation by using MTT assay method. The semi-synthetic analogues also displayed induction of apotosis in HL-60 leukemia cells as estimated by flow cytometry. The induction of apoptosis in HL-60 cells was also validated by generation of DNA fragmentation. Boswellic acid analogues of formula 1 also displayed early generation of reactive oxygen species as well as endogenous generation of nitric oxide measured by flow cytometric methods. In an animal model, the compound of formula 1 also demonstrated regression of Ehrlich Ascitic Tumor in mice.

In vitro cytotoxic acivity of natural boswellic acids on human cancer cell lines

| | | | | | | | |
|---|---|---|---|---|---|---|---|
| | | HT-29 | SW-620 | Colo-205 | | DU-145 | |
| Compound | Conc. | Colon | Colon | Colon | Hep-2 | Lung | PC-3 |

Growth inhibition %

| | | | | | | | |
|---|---|---|---|---|---|---|---|
| 1, R₃ = H, R₁ = H, R₄ = R₅ = H | 5 × 10⁻⁵ | 69 | 61 | 86 | 45 | 95 | 100 |
| Boswellic acid | | | | | | | |
| 1, R₃O = —COCH₃, R₁ = H, | 5 × 10⁻⁵ | 96 | 97 | 95 | 88 | 0 | 45 |
| R₄ = R₅ = H | | | | | | | |
| Acetyl Boswellic acid | | | | | | | |
| 1, R₃ = H, R₁ = H, R₄ = R₅ = O | 5 × 10⁻⁵ | 38 | 28 | 50 | 34 | 101 | 100 |
| 11-keto-boswellic acid | | | | | | | |
| 1, R₃O = —COCH₃, R₁ = H, | 5 × 10⁻⁵ | 91 | 91 | 89 | 62 | 88 | 79 |
| R4 + R5 = O | | | | | | | |
| Acetyl-11-keto-boswellic acid | | | | | | | |

A bioactive triterpenoid of formula 1 in the pharmaceutical preparation inhibits human breast MCF-7 cancer cells growth by 50% at about 15 μg/ml concentration. A bioactive product of formula 1 comprising triterpenoids kills human cancer cells by induction of apoptosis. A bioactive product of formula 1 kills more than 92% of prostrate DU-145 and PC-3 cells at 5×10⁻⁵M concentration, wherein the prostrate cell lines are selected from DU-145 and PC-3 cells. A bioactive product of formula 1 kills colon cancer cells up to 96% at 5×10⁻⁵M concentration, wherein the colon cancer cell lines are selected from HT-29, SW-620 and Colo205 cells. A bioactive product of formula 1 inhibits growth of cancer cells of liver up to 92% at 5×10⁻⁵M concentration, wherein the cancer cell line of liver Hep2 is selected.

The following examples are given by way of illustration and therefore should not be construed to limit the scope of the present invention.

### EXAMPLES

### Example-1

### Screening of Cell Cytotoxicity in a Panel of Human Cancer Cell Lines In Vitro

### Influence of Boswellic Acid Analogs

Assay of cytotoxicity potential of test materials is a primary standard procedure for seeking lead molecules for development of anti-cancer leads. Human cancer cells after trypsinization into single cell suspension were grown in 96-well culture plate for 24 hr. Cells were treated with indicated doses of test analogues and incubated in CO₂ incubator for 48 hr. Thereafter, cells were stained with sulforhodamine B dye, and the bound dye was eluted to measure the optical density indicating cell growth in Elisa Reader at 540 nm [Monks et al., 1991]. The OD of untreated cells is considered as 100% while of boswellic acid analogs-treated groups are subtracted from the control group to determine percent inhibition as a measure of cell cytotoxicity.

TABLE 1
In vitro cytotoxic activity of boswellic acid analogues on human cancer cell lines

| | | | | | | | |
|---|---|---|---|---|---|---|---|
| | | HT-29 | SW-620 | Colo-205 | | DU-145 | |
| Compound | Conc. | Colon | Colon | Colon | Hep-2 | Lung | PC-3 |

Growth inhibition %

| | | | | | | | |
|---|---|---|---|---|---|---|---|
| 1, R₃ = H, R₁ = H, R₄ = R₅ = H | 5 × 10⁵ | 73 | 65 | 85 | 66 | 92 | 69 |
| 1, R₃O = —COCH₃, R₁ = H, R₄ = R₅ = H | 5 × 10⁵ | 34 | 38 | 34 | 41 | 89 | 54 |
| 1, R₃O = —COCH₂CH_{3,} R₁ = H, R₄ = R₅ = H | 5 × 10⁵ | 94 | 94 | 86 | 86 | 86 | 98 |
| 1,R₃O = COCH₂CH₂CH_{3,} R1 = H, R4 = R5 = H | 5 × 10⁵ | 54 | 83 | 71 | 65 | 85 | 80 |
| 1, R = H, R₁ = H, X = O | 5 × 10⁵ | 46 | 49 | 52 | 53 | 86 | 68 |
| 1, R₃O = COCH₃, R₁ = H, R4 + R5 = O | 5 × 10⁵ | 45 | 48 | 56 | 50 | 85 | 60 |
| 1, R = COCH₂CH_{3,} R₁ = H, R4 + R5 = O | 5 × 10⁵ | 67 | 75 | 80 | 65 | 93 | 99 |
| 1, R₃O = COCH₂CH₂CH_{3,} R₁ = H, R4 + R5 = O | 5 × 10⁵ | 60 | 83 | 94 | 68 | 95 | 100 |
| 1, R = COCH₂CH_{3,} R₁ = H, R₄ = R₅ = H | 5 × 10⁵ | 97 | 99 | 87 | 90 | 96 | 96 |
| 1, R₃O = COCH₂CH₂CH_{3,} R1 = H, R4 = R5 = H | 5 × 10⁵ | 98 | 101 | 91 | 99 | 92 | 75 |
| 1, R₃O = COCH₂CH_{3,} R₁ = H, R₄ + R₅ = O | 5 × 10⁵ | 92 | 90 | 92 | 72 | 26 | 61 |
| 1, R₃O = COCH₂CH₂CH_{3,} R₁ = H, R₄ + R₅ = O | 5 × 10⁵ | 98 | 99 | 94 | 99 | 93 | 98 |
| 1, R₃O = COH, R₁ = H, R₄ = R₅ = H | 5 × 10⁵ | 79 | 92 | 93 | 75 | 94 | 99 |
| 1, R₃O = COH, R₁ = H, R₄ + R₅ = O | 5 × 10⁵ | 62 | 70 | 76 | 56 | 87 | 99 |
| 1, R₃O = —CO(CH₂)₄CH₃, R₁ = H, R₄ + R₅ = O | 5 × 10⁵ | 96 | 100 | 95 | 100 | 80 | 73 |
| 1, R₃ = H, R₁ = Me, R₄ = R₅ = H | 5 × 10⁵ | 33 | 49 | 40 | 26 | 88 | 57 |
| 1, R₃ = H, R₁ = Me, R₄ + R₅ = O | 5 × 10⁵ | 70 | 70 | 73 | 52 | 68 | 85 |
| 1,R₃O = —COCH₂CH₂COOH, R₁ = H, R₄ = R₅ = H | 5 × 10⁵ | 71 | 71 | 92 | 58 | 96 | 87 |
| 1,R₃O = —COCH₂CH₂COOH, R₁ = H, R₄ + R₅= O | 5 × 10⁵ | 3 | 0 | 1 | 7 | 50 | 38 |
| 1,R₃O = —COCH₂CH₂COOH, R₁ = H, R₄ = R₅ = H | 5 × 10⁵ | 11 | 1 | 20 | 10 | 50 | 38 |
| 1,R₃O = —COCH₂CH₂COOH R₁ = H, R₄ + R₅ = O | 5 × 10⁻⁵ | 16 | 2 | 29 | 17 | 87 | 60 |
| 1, R₃ = H, R₁ = Me, R4 = R5 = H | 5 × 10⁻⁵ | 4 | 9 | 20 | 0 | 2 | 4 |
| 1, R₃ = H, R₁ = Me, R₄ + R₅ = O | 5 × 10⁻⁵ | 7 | 11 | 20 | 5 | 51 | 8 |

TABLE 2
Comparative in vitro cytotoxic acivity of standard
anticancer drugs on human cancer cell lines

| | | | | | | | |
|---|---|---|---|---|---|---|---|
| | | HT-29 | SW-620 | Colo-205 | | DU-145 | |
| Compound | Conc. | Colon | Colon | Colon | Hep-2 | Lung | PC-3 |

Growth inhibition %

| | | | | | | | |
|---|---|---|---|---|---|---|---|
| 5-FU | 1 × 10⁵ | 40 | 0 | 43 | 14 | 0 | 37 |
| 5-FU | 1 × 10⁴ | 21 | 7 | 39 | 5 | 20 | 52 |
| Mitomycin C | 1 × 10⁷ | 19 | 27 | 41 | 12 | 5 | 18 |
| Mitomycin C | 1 × 10⁶ | 33 | 57 | 28 | 46 | 29 | 24 |

### Example-2

### Inhibition of Cell Proliferation by Structural Analogs of Boswellic Acid

### MTT Assay

Human leukemia cells HL-60 were grown in suspension in 96-well culture plate and were incubated with different concentrations of the test analogs for 48 hr. The cells were then incubated with MTT and the MTT-formazon formed is eluted with DMSO, and OD measured in ELISA Reader [Shashi et al., 2006]. The intensity of the color formed in the untreated control wells relates to 100% cell growth. The growth of cells is recorded with different concentrations of the structural analogs, and the concentration that inhibits 50% cell growth is taken as IC₅₀ value. The IC50 values in HL-60 cells were between 10-15 μM.

### Example-3

### Concentration Related Influence of Boswellic Acid Analogs on the Relative Degree of Inducibility of Apoptosis

### Flow Cytometric Analysis in HL-60 Leukemia Cells

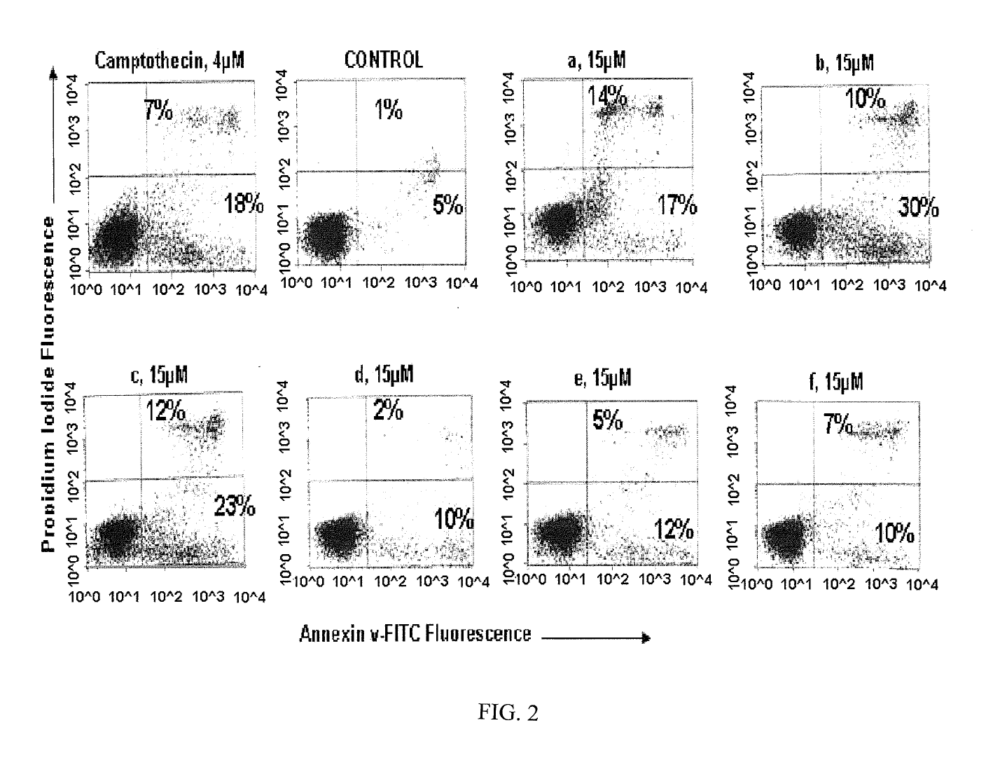

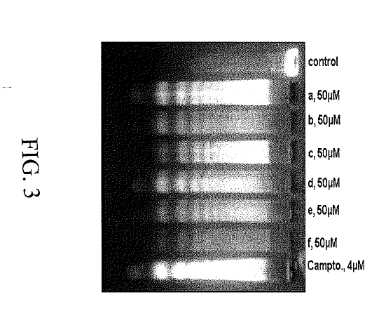

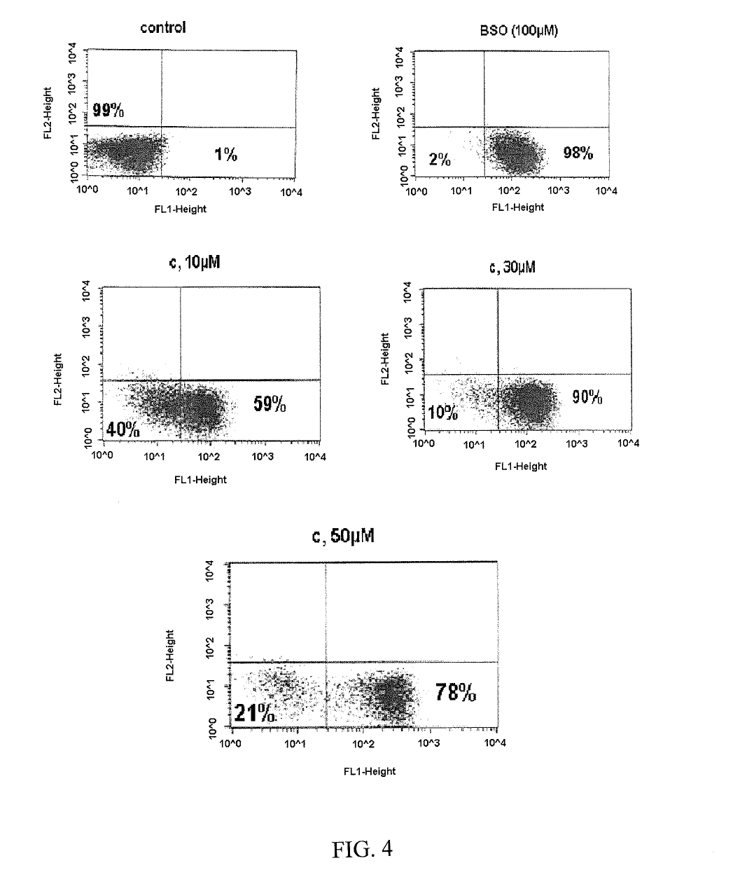

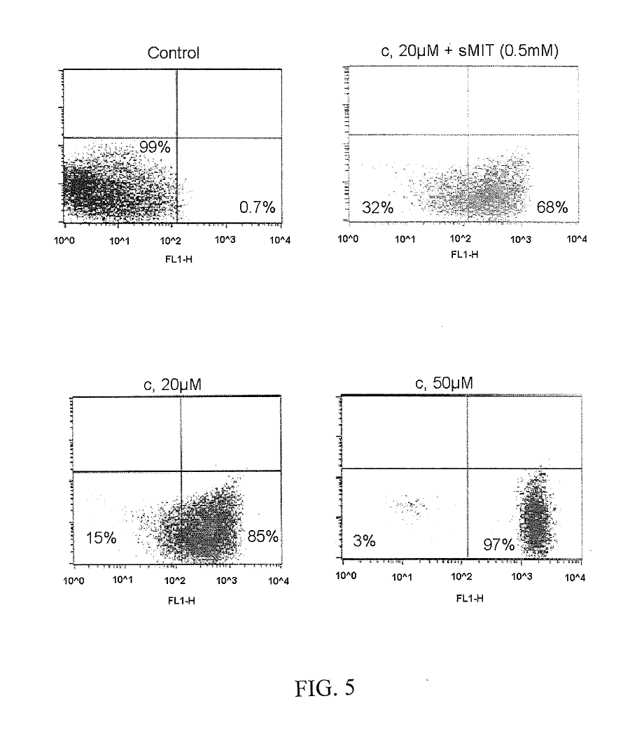

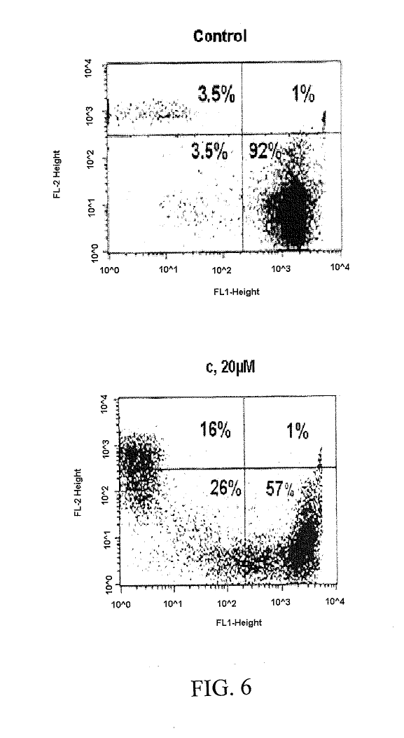

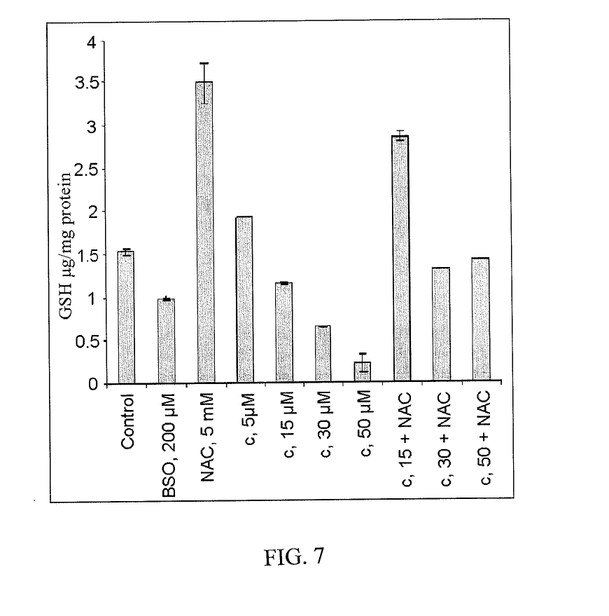

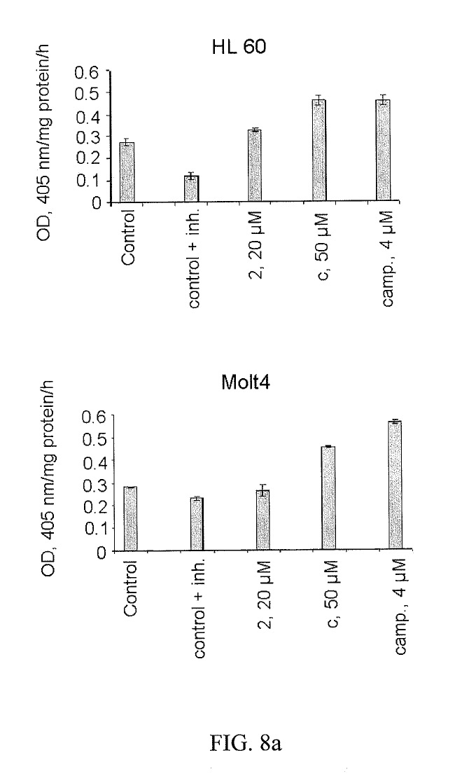

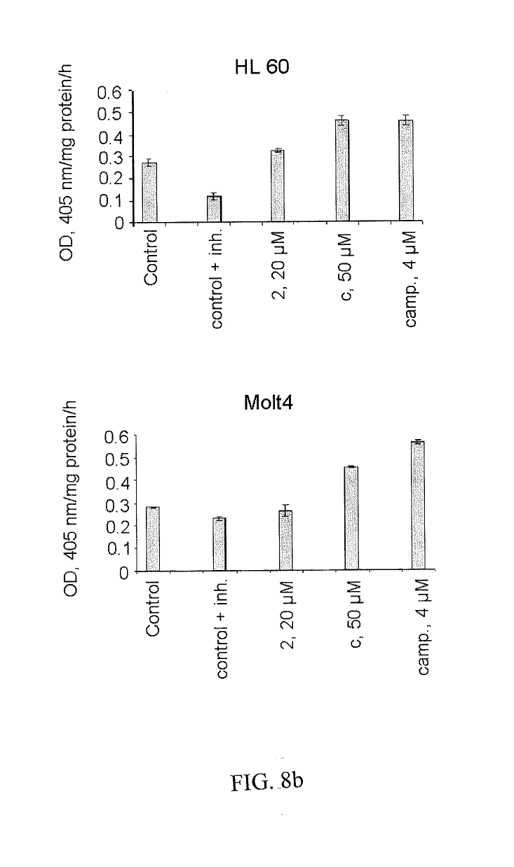

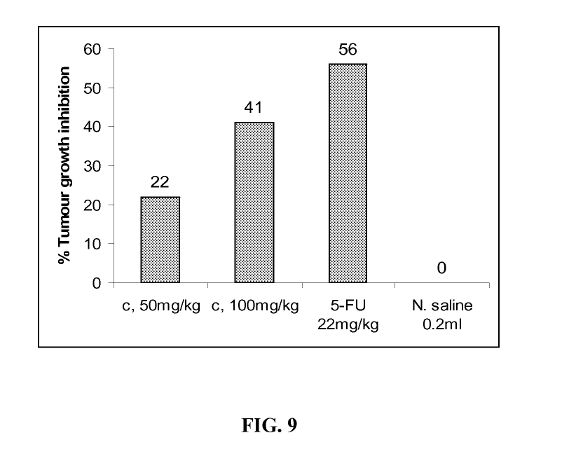

During the early events of apoptosis, phospholipid phosphatidyl serine of plasma membrane is externalized, which has very high affinity for annexinV antibody. Effect of a single concentration (15 μM) of each boswellic acid analogs on the relative efficiency of induction of apoptosis and necrosis in HL-60 cells was analyzed by flow cytometry. Cells were incubated with each analog for 6 hr and stained with Annexin V-FITC/PI. Camptothecin at 4 μM was used as positive control. There after, cells were washed and stained with FITC conjugated annexinV antibody and propidium iodide. The cells (10,000) were analysed by flow cytometery (BD, LSR) using ProQuest software. As shown in FIG. 2, the fraction of cell population in the lower right quadrant indicates apoptotic cells, upper right post-apoptotic and upper left as necrotic populations (FIG. 2).

### Example-4

### Validation of Apoptosis by Generation of DNA Fragmentation Typical of Apoptosis in HL-60 Cells

The structural analogs produced programmed cell death (apoptosis), a desired therapeutic anticancer drug target in human leukaemia HL-60 cells. Human leukemia cells were grown in culture and exposed to 50 uM concentration of each analog for 6 hr. The control group received only the vehicle. Cells were similarly treated with camptothecin, 4 μM as positive control. Genomic DNA was extracted and electrophoresed on agarose gel [Sashi et al., 2006].

### Example-5

### Influence of Boswellic Acid Analogs on the Early Generation of Reactive Oxygen Species (Peroxides)

### Analysis by Flow Cytometery

The endogenous generation of peroxides was measured using a non-fluorescent probe DCFH-DA which upon entering the cell is deesterified to DCFH which is oxidized by the reactive oxygen species to a fluorescent product DCF that remains entrapped within the cells offering analysis by flow cytometery [Shashi et al, 2006]. HL-60 cells were incubated with different concentrations of compound of formula 1 where R₃=COCH₂CH₂CH₃, R₁=H, R₄+R₅=O for a brief period of 6 hr. Thereafter cells were washed and stained for 1 hr with DCFH-DA and analysed on flow cytometer. The analog produced dose dependent increase in peroxide positive cell population, being 79% at 50 μg/ml. The pro-oxidant effect was completely impaired in the presence of anti-oxidant, ascorbate. Formation of reactive oxygen and nitrogen species are indicated in the induction of apoptosis.

### Example-6

### Compound of Formula 1 where R₃=COCH₂CH₂CH₃, R₁=H, R₄+R₅=O Mediated Early Generation of Endogenous Nitric Oxide Measured by Flow Cytometery

Measurement of in situ NO generation involved the use of a fluorescent probe diaminofluoresceine-2-diacetate, which is permeated easily into the cells. Once inside the cell it binds NO soon it is formed and emits fluorescence [H. Kojima et al, 1998]. For this purpose, the HL-60 cells were incubated with DAF-2-DA for 30 min before being incubated with the indicated concentration of SS-145 for 4 hr. Cells were analyzed on flow cytometer in FL-1 channel for NO. It appears that all the cells formed NO within 6 hr irrespective of the concentration of bioactive compound used, because there were no cells in the lower left quadrant as indicated in control cells. Interaction of NO with superoxide, while both are generated simultaneously, may affect the mitochondrial membrane potential that may be responsible for the activation of apoptosis on account of oxidative stress produced by compound of formula 1 where, R₃=COCH₂CH₂CH₃, R₁=H, R₄+R₅=O.

### Example-7

### Mitochondrial Membrane Depolarization by a Compound of Formula 1 where R₃=COCH₂CH₂CH₃, R₁=H, R₄+R₅=O

### Measurement by Flow Cytometery

Rhodamine-123 uptake into the mitochondria is driven by mitochondrial transmembrane potential (ψₘₜ) that allows the determination of cell population with active integrated mitochondrial functions. Loss of ψₘₜ would lead to depolarization of mitochondria because of ROS and NO generation leading to cell death. HL-60 cells were exposed to SS-145 for 12 hr and incubated with Rh-123. Cells, 10000, were acquired for analysis by flow cytometery. The percentage of cells with low Rh-123 fluorescence was calculated from the dot plot statistics. In untreated control cells more than 92% cells showed Rh-123 fluorescence, which decreased with the treatment of compound of formula 1 where R₃=COCH₂CH₂CH₃, R₁=H, R₄+R₅=O, approx. 42% at 20 μM concentration. This shows that reactive oxygen/nitrogen species generated by compound of formula 1 bring about oxidative damage to mitochondria ensuing apoptosis.

### Example-8

### A Compound of Formula 1 where R₃=COCH₂CH₂CH₃, R₁=H, R₄+R₅=O Produced Concentration Dependent Depletion of Reduced Glutathione Contents in HL-60 Cells

Reduced glutathione content was determined in 3×10⁶ HL-60 cells/well/3 mL medium in a 6-well plate after incubation with SS-145 for 6 hr. Cells were washed and the reduced glutathione contents were extracted and determined using a standard fluorimetric method [Hissen et al., 1976]. Cells were also treated with BSO (200 μM) as positive control, and NAC (5 mM) as anti-oxidant.

### Example-9A

### Influence of Compound of Formula 1 where R₃=COCH₂CH₂CH₃, R₁=H, R₄+R₅=O on the Activation of Caspases in Leukemia Cells

Caspases are important cysteine-proteases that are involved in the cleavage of DNA specifically producing 180 bp DNA fragments. The enzymes act through different signaling pathways. Caspase-9 activation involves mitochondrial mediated pathway initiates apoptotic process by recruiting caspase-3 for the execution of programmed cell death. Caspase-9 activity was determined fluorometrically and caspase-3 colorimetrically using kits and the instruction provided by the manufacturer (BD pharmingen, USA). Caspase-3 activity was determined in both Molt-4 and HL-60 leukemia cells while caspase-9 was determined in HL-60 cells.

A compound of formula 1 where R₃=COCH₂CH₂CH₃, R₁=H, R₄+R₅=O was used as the template for other analogs. The analog activated caspase-3 by about 50% when cells were incubated with the analog for 6 hrs. The activity was almost abolished in the presence of selective inhibitor demonstrating that the activity is solely of the caspase-3.

Activation of caspase-3 in leukemia cell lines by boswellic acid analog. Caspase-3 is an executioner enzyme guiding the fragmentation of DNA and its activity was stimulated by about 50% when both leukemia cells were incubated with compound of formula 1 where R₃=COCH₂CH₂CH₃, R₁=H, R₄+R₅=O for 6 hr (FIG. 8a). The specific inhibitor used decreased the activity suggesting that the protease measured is certainly caspase-3 activity.

### Example. 9B

### Compound of Formula 1 where R₃=COCH₂CH₂CH₃, R₁=H, R₄+R₅=O Stimulated Strongly the Caspase-9 Activity in HL-60 Cells

Caspase-9 activity is very sensitive to oxidative stress and as a result its activity is stimulated by 4-5-fold at 50 μM compound of formula 1 where R₃=COCH₂CH₂CH₃, R₁=H, R₄+R₅=O (FIG. 8b) when at this concentration the caspase-3 was stimulated by about 50% only. Caspase-9 activations suggests the leakage of cytochrome c from mitochondria and activation of several genes and downstream signal transduction pathways leading to the cleavage of DNA mediated by caspase-9.

### Example-10

### Effect of Compound of Formula 1 where R₃=COCH₂CH₂CH₃, R₁=H, R₄+R₅=O on the Regression of Ehrlich Ascitic Tumor in Mice

Swiss albino mice of 6-8 weeks, weighing 18-23 g of single sex were used. For the initiation of experiment, 1×10⁷ cells of EAC (Ehrlich Ascitic Carcinoma) cells were transplanted intramuscularly (i.m.) in 32 mice for the development of solid tumors. The day of tumor transplantation was assigned as day 0. Next day (day-1) animals were randomly selected and divided into four groups having equal number of mice in each group of similar body wt. Groups I and II were treated with compound of formula 1 where R₃=COCH₂CH₂CH₃, R₁=H, R₄+R₅=O at the dose of 50 mg/kg b, wt and 100 mg/kg b. wt. intraperitoneally, respectively. Group III received 0.2 ml of 0.85% normal saline and served as normal control while group IV serving as positive control was treated with 5-FU (22 mg/kg b. wt.) intraperitoneally. The total duration of treatment was of 9 days. The evaluation of effect of treatment on tumor was done on 13^{th} day. Ehrlich ascites cells (1×10⁷) were injected intramuscularly in the right thigh muscles and on the following day animals started receiving treatment intraperitoneally in 0.5% Tween-20 daily at the indicated doses for 9 days. The growth of tumor and the efficacy of compound of formula 1, where R₃=COCH₂CH₂CH₃, R₁=H, R₄+R₅=O in inhibiting tumor growth were evaluated on day 13 in terms of the weight of the tumor in treated and control groups count. The determination of solid tumor growth inhibition against EAT was calculated as percent tumor growth regression according to the following:

$$Tumor growth regression ( \% ) = \frac{\begin{matrix}avg . tumor weight of control - \\ avg . tumor weight of treated\end{matrix} }{Average tumor wt of control} \times 100$$

$$where tumor weight ( mg ) = [ ( \underline{Length \times {( width )}^{2}} ] / 2$$

### REFERENCES

### Patents

Indian Patent 178627 dated 16 Jan. 1998
Indian Patent 180492 dated. 8 Jan. 1999
U.S. Pat. No. 5,919,821
U.S. Pat. No. 6,174,876
U.S. Patent Application 20020010168
U.S. Patent Application 20050084547
Ammon, H. P. T. et al., “Mechanism of antiinflammatory actions of curcumine and boswellic acids,” J Ethnopharmacol. 1993 March; 38(2-3):113-9.
Ammon, H. P. T., “Boswellic acids (components of frankincense) as the active principle in treatment of chronic inflammatory diseases,” Wien Med Wochenschr. 2002; 152(15-16):373-8. Review. German.;
G. Minotti et al.; “Anthracyclines: Molecular advances and pharmacologic developments in anti-tumor activity and cardiotoxicity,” Pharmacol. Rev. 56 (2004), 185-229.
Glaser T, et al., “Boswellic acids and malignant glioma: induction of apoptosis but no modulation of drug sensitivity,” Br. J. Cancer, 1999, 80, 756-65;
Gupta, I. et al., “Effects of Boswellia serrata gum resin in patients with ulcerative colitis,” Eur J Med Res. 1997 January; 2(1):37-43;
Gupta, I. et al., “Effects of gum resin of Boswellia serrata in patients with chronic colitis.” Planta Med. 2001 July; 67(5):391-5;
H. Kojima et al., “Development of a fluorescent indicator for the nitric oxide based on the fluoresceine chromophore,” Chem. Pharm. Bull., 46 (1998) 373-375.
Hissen P. J. and Hilff, R. (1976) A fluorometric method for determination of oxidized and reduced glutathione in tissues, Analytical Biochemistry, 74, 214-226
Hoernlein, R. F. et al., “Acetyl-11-keto-β-boswellic acid induces apoptosis in HL-60 and CCRF-CEM cells and inhibits topoisomerase I,” J. Pharmacol. Exp. Ther., 1999, 288, 613-19;
Hostanska K. et al., “Cytostatic and apoptosis-inducing activity of Boswellic acids toward malignant cell lines in vitro.” Anticancer Res 2002; 22:2853-62.
Janssen G, et al., “Boswellic acids in the palliative therapy of children with progressive or relapsed brain tumors,” Klin. Padiatr. 2000, 212, 189-95.
Jian-Jun Liu et al., “Boswellic acids trigger apotosis via a pathway dependent on caspase-8 activation but independent of Fas/Fas ligand interaction in colon cancer HT-29 cells,” Carcinogenesis, 2002, 23(12), 2087-93;
Jian-Jun Liu et al., “Keto and acetyl keto boswellic acids inhibit proliferation and induce apoptosis in HepG2 cells via a capsase-8 dependent pathway,” Int. J. Mol. Med., 2002, 10, 501-05;
K. M. Deabatin, “Activation of apoptosis pathways by anti-cancer treatment,” Toxicol. Lett., 112/113 (2000) 41-48.
Kapil, A. et al., “Anticomplementary activity of boswellic acids—an inhibitor of C3-convertase of the classical complement pathway,” Int J Immunopharmacol. 1992 October; 14(7):1139-43.
Kreiglstein, C. F. et al., “Acetyl-11-keto-beta-boswellic acid, a constituent of a herbal medicine from Boswellia serrata resin, attenuates experimental ileitis,” Int J Colorectal Dis. 2001 April; 16(2):88-95;
Lee, et al., (1991) U.S. Pat. No. 5,064,823;
Monks, et al., “Feasibility of a high-flux anticancer drug screen using a diverse panel of cultured human tumor cell lines,” J. Natl. Cancer Inst. 83 (1991) 757-766.
Roy, S. et al., “Human genome screen to identify the genetic basis of the anti-inflammatory effects of Boswellia in microvascular endothelial cells,” DNA Cell Biol. (2005) April; 24(4):244-55.
Safayhi, H. et al., “Boswellic acids: novel, specific, nonredox inhibitors of 5-lipoxygenase,” J Pharmacol Exp Ther. 1992 June; 261(3): 1143-6.
Schweizer, S. et al., “Workup-dependent formation of 5-lipoxygenase inhibitory activity of boswellic acid analogues,” J Nat Prod. 2000 August; 63(8): 1058-61;
Shao et al., Planta Medica 64 (1998) 328-31.
Sharma, M. L. et al., “Anti-arthritic activity of boswellic acids in bovine serum albumin (BSA)-induced arthritis,” Int J Immunopharmacol. 1989; 11(6):647-52.
Shashi B., Singh, J., Rao, J. M., Saxena, A. K. and Qazi, G. N. (2006), “A novel lignan composition from Cedrus deodara induces apoptosis and early nitric oxide generation in human leukemia Molt-4 and HL-60 cells,” Nitric Oxide, 14, 72-88)
Streffer, J. R. et al., “Response of radiochemotherapy-associated cerebral edema to a phytotherapeutic agent, H15,” Neurology, 2001, 56, 1219-21.
Syrovets et al., “Acetyl-boswellic acids are novel catalytic inhibitors of human topoisomerases I and II alpha,” Mol. Pharmacol., 2000, 58, 71-81.
Syrovets et al., “Inhibition of IkB Kinase activity by acetyl-β-boswellic acids promotes apotosis in androgen-independent PC-3 Prostate cancer cells and in vivo,” J. Biol. Chem., 2005, 280(7), 6170-80;
Wanzhou Zhao et al., “Boswellic acid acetate induces differentiation and apoptosis in highly metastatic melanoma and fibrosarcoma cells,” Cancer Detection and Prevention, 2003, 27(1), 67-75;
Yongkui Jing et al., “Boswellic acid acetate induces differentiation and apoptosis in leukemia cell lines,” Leukemia Research, 1999, 23, 43-50;

1. A method comprising:
obtaining a compound derivable semi-synthetically from boswellic acids of formula 1: 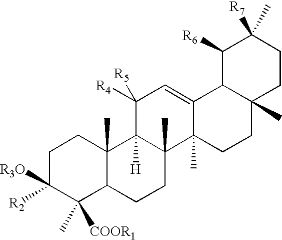
wherein R₁ is H or Me; R₂=R₃=H or acyl group with C₂-C₅ alkyl chain; R₄=R₅=H or R₄+R₅=O; R₆=R₇=H or Me; and
contacting a cell with the compound.

2. The method of claim 1, wherein the compound has anticancer activity.

3. The method of claim 2, wherein the compound is:
3α-hydroxyurs-12-ene-24-oic acid;
3α-hydroxyolean-12-ene-24-oic acid;
11-keto-3α-hydroxyurs-12-ene-24-oic acid;
11-keto-3α-hydroxyolean-12-ene-24-oic acid;
3β-hydroxyurs-12-ene-24-oic acid;
3β-hydroxyolean-12-ene-24-oic acid;
11-keto-3β-hydroxyurs-12-ene-24-oic acid; or
11-keto-3β-hydroxyolean-12-ene-24-oic acid.

4. The method of claim 2, wherein the compound shows cytotoxicity and anti-cancer activity up to 100% for colon, prostate, liver breast, central nervous system (CNS), leukemia, and/or malignancy of other tissues, kidney, lung, muscle, ovarian, and/or prostate cancer.

5. The method of claim 2, wherein the compound shows cytotoxicity and growth inhibition up to 52% of Ehrlich Ascites tumors and solid tumors.

6. The method of claim 2, wherein the compound kills up to 99% of prostrate cancer cells from one or more of DU-145 and PC-3 cell lines at 5×10⁻⁵M concentration.

7. The method of claim 2, wherein the compound kills colon cancer cells from one or more of HT-29, SW-620, and Colo205 cell lines up to 100% at 5×10⁻⁵M concentration.

8. The method of claim 2, wherein the compound inhibits growth of cancer cells of liver Hep2 up to 100% at 5×10⁻⁵M concentration.

9. The method of claim 2, wherein cancer cell death is mediated by induction of apoptosis and inhibition of cell proliferation.

10. The method of claim 2, wherein the compound is capable of producing early reactive nitrogen species nitric oxide up to 80% and acting as causative agents ensuing DNA laddering and apoptotic death of cancer cells.

11. The method of claim 2, wherein the compound is capable of inducing at least up to 50% mitrocondrial depolarization consequent to reactive oxygen/nitrogen species as the mechanisms in to the apoptotic death of cancer cells.

12. The method of claim 2, wherein the cell is in a subject.

13. The method of claim 12, wherein the subject is a human

14. The method of claim 12, wherein the compound is administered at a concentration of 5-100 μg/ml.

15. The method of claim 12, wherein a therapeutically effective dose of the compound is in the range of 5×10⁻⁵-100×10⁻⁵ M.

16. The method of claim 15, wherein the therapeutically effective dosage of is administered to a subject for 5-15 days.

17. The method of claim 12, wherein the administration is oral, intra-venous, intra-peritoneal, or nasal.

18. The method of claim 12, wherein the composition is non-toxic in the range of 250-1000 μg/kg body weight of the subject.

19. A pharmaceutical composition comprising a compound derivable semi-synthetically from boswellic acids of formula 1: 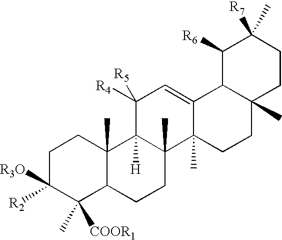
wherein R₁ is H or Me; R₂=R₃=H or acyl group with C₂-C₅ alkyl chain; R₄=R₅=H or R₄+R₅=O; R₆=R₇=H or Me.

20. The pharmaceutical composition of claim 19, wherein the compound is at a concentration of 5-100 μg/ml.
